(19) United States
(12) Patent Application Publication
(10) Pub. No.: US 2026/0283513 A1
(43) Pub. Date: Sep. 24, 2026

(54) SYSTEM FOR MONITORING FETAL STATUS DURING CHILDBIRTH

(71) Applicant: HeraLife Medical, Inc., Wyndmoor, PA (US)

(72) Inventor: John S. Missanelli, Wyndmoor, PA (US)

(73) Assignee: HeraLife Medical, Inc., Wyndmoor, PA (US)

(21) Appl. No.: 19/686,437

(22) Filed: May 22, 2026

Related U.S. Application Data
(63) Continuation of application No. 18/342,143, filed on Jun. 27, 2023, now Pat. No. 12,642,463.
Application No. 18/342,143 is a continuation-in-part of application No. 18/299,027, filed on Apr. 11, 2023, now abandoned.
(60) Provisional application No. 63/355,672, filed on Jun. 27, 2022.
(60) Provisional application No. 63/355,673, filed on Jun. 27, 2022.
(60) Provisional application No. 63/355,675, filed on Jun. 27, 2022.
(60) Provisional application No. 63/355,677, filed on Jun. 27, 2022.
(60) Provisional application No. 63/355,679, filed on Jun. 27, 2022.
(60) Provisional application No. 63/355,681, filed on Jun. 27, 2022.
(60) Provisional application No. 63/355,682, filed on Jun. 27, 2022.
(60) Provisional application No. 63/355,684, filed on Jun. 27, 2022.

(51) Int. Cl. A61B 5/1464; A61B 5/00; A61B 5/0205; A61B 5/1455

(52) U.S. Cl. CPC A61B 5/1464; A61B 5/0205; A61B 5/14551; A61B 5/4362; A61B 2503/02; A61B 2562/0238

(57) ABSTRACT

During childbirth process, trauma to an infant can readily arise, ultimately resulting in fetal hypoxia, academia, and brain damage. Such unfavorable conditions can be prevented by measuring the fetus' blood-oxygen level and heart rate. Without a fetal pulse oximeters, blood oxygen level cannot be monitored non-invasively reliably, which reduces the chance for birth complications to be recognized in time. A noninvasive system to implement such goals and maximize the potential welfare of the fetus may include devices to measure oxygen saturation of hemoglobin (SpO2). Such a device may be an oxy probe that uses a trans-reflective method of SpO2 measurement where oxygen saturation data can be transmitted through wire, fiber optics, and or using a radio frequency link, fetal monitor data can be analyzed, compared to existing data base, and or transmitted via radio waves or internet.

### CROSS-REFERENCE TO RELATED APPLICATIONS

This application is a continuation of, and claims priority under 35 U.S.C. § 120 to, U.S. patent application Ser. No. 18/342,143, filed Jun. 27, 2023, now allowed and scheduled to issue as U.S. Pat. No. 12,642,463 on Jun. 2, 2026, which is a continuation-in-part of U.S. patent application Ser. No. 18/299,027, filed Apr. 11, 2023, now abandoned, and which further claims the benefit under 35 U.S.C. § 119(e) of U.S. Provisional Patent Application Nos. 63/355,672; 63/355,673; 63/355,675; 63/355,677; 63/355,679; 63/355,681; 63/355,682; and 63/355,684, each filed Jun. 27, 2022. U.S. patent application Ser. No. 18/299,027 is, in turn, a continuation of U.S. patent application Ser. No. 16/540,615, filed Aug. 14, 2019, now abandoned, which claims the benefit under 35 U.S.C. § 119(e) of U.S. Provisional Patent Application No. 62/718,754, filed Aug. 14, 2018. The entire disclosures of each of the foregoing applications are incorporated herein by reference.

### BACKGROUND

Pulse oximeters have conventionally been used to measure the oxygen saturation of arterial blood continuously. To use the pulse oximeters, a probe is attached to the tip of a subject's finger or earlobe, and both red and the probe applies infrared light having different wavelengths to the living body from the probe at given time intervals. The oximeter calculates the oxygen saturation from the ratio between the RED and IR of light absorbance. In a typical case, the red light has a reference wavelength of 660 nm and the infrared light has a wavelength of 900 nm; two light-emitting diodes of these wavelengths and one photodiode for light reception may be contained in the probe.

Although fetal heart monitors can be used as a surrogate means to attempt to measure fetal blood oxygen saturation levels, this method is indirect and thus does not give a fully complete understanding of the fetal status. As a result of this lack of full understanding, emergency medical decisions, such as when to start an emergency caesarean section (C-section) must be made with incomplete knowledge. As a practical matter, doctors sometimes err on the side of caution, which may result in unnecessary C-sections, and the attendant high medical expenses and maternal post-childbirth complications.

Previous attempts to provide this missing fetal blood oxygen saturation levels include the OxiFirst system, produced by Mallinckrodt/Nellcor, now part of Tyco Healthcare. This system, which obtained FDA approval in 2000, works by directly placing the tip of a pulse oximeter sensor up the maternal birth canal, through the cervix, into the uterus, and onto the cheek or temple of the fetus. This method is described in U.S. Pat. Nos. 5,813,980; 5,109,849, and 4,938,218 which are incorporated by reference as if fully set forth herein. Unfortunately, due to the high invasiveness and bother of the procedure, the method met with limited medical acceptance in the field, and the manufacturer eventually decided to stop selling the device.

U.S. Pat. No. 5,135,006, which is incorporated by reference as if fully set forth herein, shows a method and apparatus for monitoring the fetus in a birth canal during labor. This fetal monitor probe monitors heartbeat and does not directly measure blood oxygenation.

U.S. Pat. No. 10,415,163, which is incorporated by reference as if fully set forth herein, is based on similar pulse oximetry principal for non-invasive monitoring of fetal blood oxygenation by directing light at the abdomen of a pregnant woman, and detecting light scattered and reflected by fetal and maternal tissues back to the surface of the mother's abdomen. It may not be as accurate because of the distance between the probe and the fetus during delivery.

U.S. Pat. No. 7,469,158, which is incorporated by reference as if fully set forth herein, is also based on a similar pulse oximetry principal for non-invasive monitoring of fetal blood oxygenation but requires it to be screwed in the scalp. It presents a more invasive technology than is desirable.

U.S. Pat. No. 8,417,307, which is incorporated by reference as if fully set forth herein, relates to a transmissive type blood oximeter for measuring the oxygenation. Still, it cannot be used in case of fetal measurements.

Thus, there exists a need for a less invasive, accurate, pulse oximeter with a probe that can accurately and safely measure arterial oxygen saturation of a fetus.

### SUMMARY OF THE EMBODIMENTS

During the childbirth process, trauma to an infant can readily arise, ultimately resulting in fetal hypoxia, academia and brain damage. Such unfavorable conditions can be prevented by measuring the fetus' blood-oxygen level and heart rate. Without a fetal pulse oximeters, blood oxygen level cannot be monitored non-invasively reliably, which reduces the chance for birth complications to be recognized in time. A noninvasive system to implement such goals and maximize the potential welfare of the fetus may include devices to measure oxygen saturation of hemoglobin (SpO2) that have been available for at least 50 years. Such a device may be an oxy probe that uses a trans-reflective method of SpO2 measurement where oxygen saturation data can be transmitted through wire, fiber optics, and or using a radio frequency link, fetal monitor data can be analyzed, compared to the existing data base, and or transmitted via radio waves or internet.

### BRIEF DESCRIPTION OF THE DRAWINGS

FIG. 1 shows the oxy probe with the connector.

FIG. 2 shows a cross-section A-A of the oxy probe shown in FIG. 1.

FIG. 3 shows a partially exploded view of the oxy probe and its components.

FIG. 4 shows a depiction of the invention in use.

FIG. 5 shows a cut away view of the oxy probe tip illustrating the light path of the reflected light.

FIG. 6 shows a cutaway view of the oxy probe tip.

FIG. 7 shows an enhanced light source and collector.

FIGS. 8A and 8B show an embodiment of an oxy probe with an included camera.

FIG. 9 shows a thermocouple in use with the oxy probe.

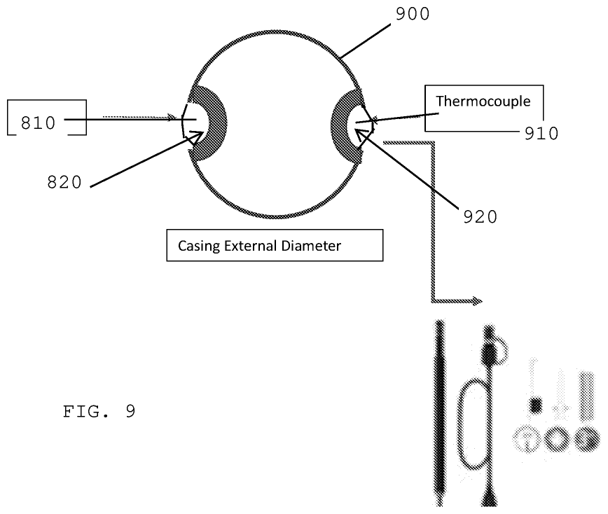

FIG. 10 shows a sample display for the oxy probe.

FIG. 11 shows a wireless pulse ox with a battery.

### DETAILED DESCRIPTION OF THE EMBODIMENTS

### Introduction

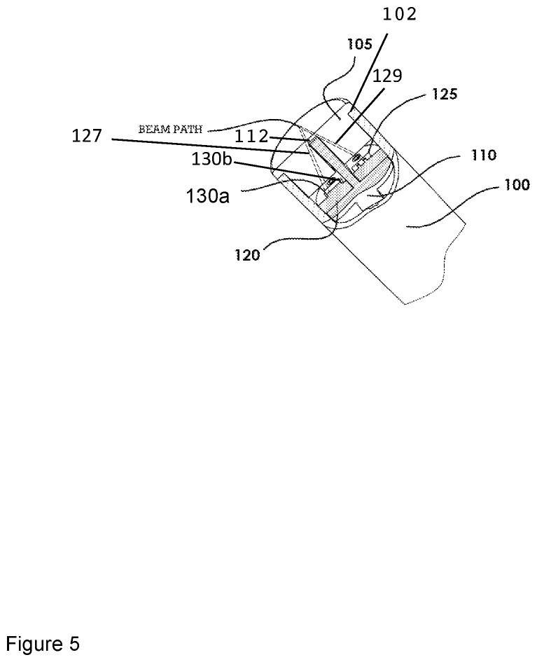

The underlying principle of operation is based on the red and infrared light absorption characteristics of oxygenated and deoxygenated hemoglobin. Oxygenated hemoglobin absorbs more infrared light and allows more red light to pass through. Deoxygenated (or reduced oxygen) hemoglobin absorbs more red light, allowing more infrared light to pass through. Red light is in the 640-720 nm wavelength light band. Infrared light is in the 840-920 nm wavelength light band. The embodiment includes a cylindrical housing 100 having two different light sources 130a, 130b, collectively 130 (if a single source), that emit emitted light 127 and a detector or sensor 125 with an opaque partition 110 between sources and detector such a way that the detector 125 will see the reflected light 129 as shown in FIG. 5. The probe 100 may include a clear tip or cap 105 that seals the tip from external fluids and at the same time allows the optical signal/light to be transmitted and received with no to minimal attenuation.

In an alternate embodiment, the probe for measuring hemoglobin oxygenation may require two wavelength emitters, Red (640-720 nm) and IR (840-920). Thus, further emitters may be used in this instance using the same probe configuration in which two other wavelengths can be added. An optional feature for detecting proximity to a fetus may be integrated into the probe.

### DETAILED DESCRIPTION

The SpO2 monitoring PROBE (called oxy probe herein) may include, as labeled:
100. Probe body or housing
105. Soft clear probe Tip
110. Probe optical divider
115. Probe wire
120. Integrated circuit IC or PC Board
125. Sensor/detector Chip
127. Emitted light.
129. Reflected or received light.
130a, 130b. Light sources
130. LED Chips
135. Probe Connector
140. Emitter Cavity
145. Sensor Cavity
200. Fetus

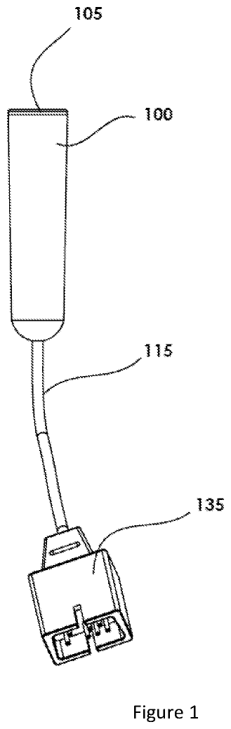

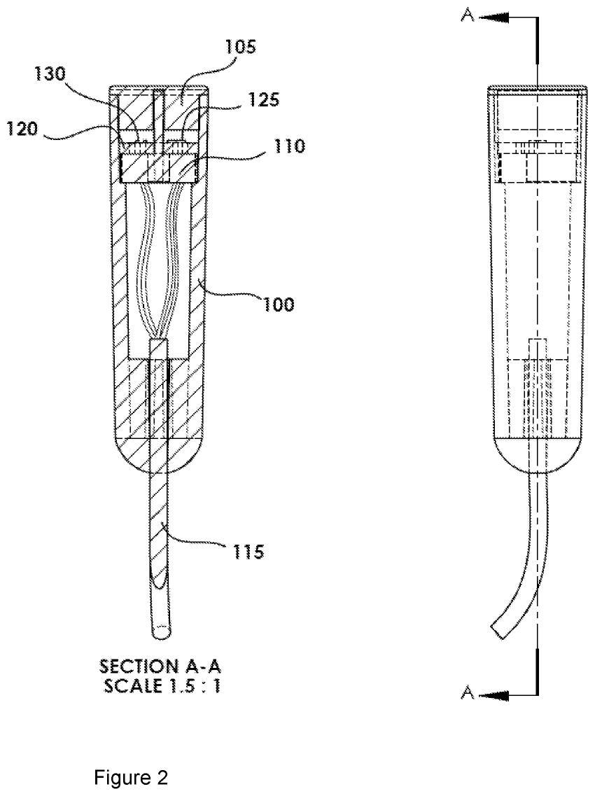

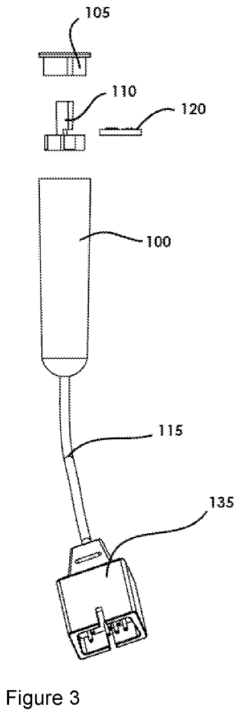

As shown in FIGS. 1-5, an intra-vaginal and intra-uterine oxy probe 100 allows the arterial oxygen saturation of the fetus 200 to be measured during childbirth process. The oxy probe 100 has a tip 105 that may be made of soft optically clear silicone-type material in its housing and tip 105, which may be safely pressed against the fetus 200 without causing any injury. The tip 105 of the oxy probe 100 is designed to have two optically isolated compartments 140 and 145: One compartment to house the emitters 140 and another to house the sensor 145, wherein the compartments may be separated by an opaque divider 110 that prevents the emitted light 127 from being directly transmitted to the sensor 145. The fetal blood pulse oximetry oxy probe 100 may use at least two wavelengths of the emitted light 127 from a first emitter 130a at about 640 to 680 nm and a second wavelength of light at about 870 to 920 nm 130b, wherein the emitters are preferably LED light emitters. The optically clear oxy probe tip 105 allows the emitted light 127 to illuminate fetal tissue and the sensor 125 can detect the reflected light 129. A CPU remote from tip 105 may perform signal processing to extract the oxygen saturation information related to fetal arterial blood.

The oxy probe may be housed in a housing 100, with dimensions of approximately 0.5 inch in diameter and 2.2-inch-long. A top 0.5 inch of the probe may include the sensor 125, emitters 130, and optical insert 110. These are nominal dimensions and can vary based on requirements, but are chosen to minimize invasiveness to the pregnant woman. The visual divider 110 may be opaque and divide the tip 105 of the probe 100 in two compartments, maintaining the optical isolation between the emitter cavity 140 and sensor 145 cavity (compartments) such that the only way for light to pass between the compartments is through reflection off another surface. The tip of the probe 100 may have a clear, soft, flexible elastomeric material lens 105 that extends into the cavities 140, 145 in such a way as to act as a seal to prevent any fluids from entering the cavities 140, 145 (FIG. 5).

This oxy probe 100 may accommodate additional sensors for monitoring a patient's other conditions, including an arterial hemoglobin oxygen saturation sensor. Most common pulse oximeters used in the hospitals are of transmittance type, whereas the emitters 130a, 130b are placed on one side and the light goes through the tissue to the sensor 125 on the opposite side of the tissue. Alternatively, the emitter 130 and sensor 125 components used in both cases may be similar, with the difference that in the reflective probe, the light reflected by the tissue is compared to light going through the tissue.

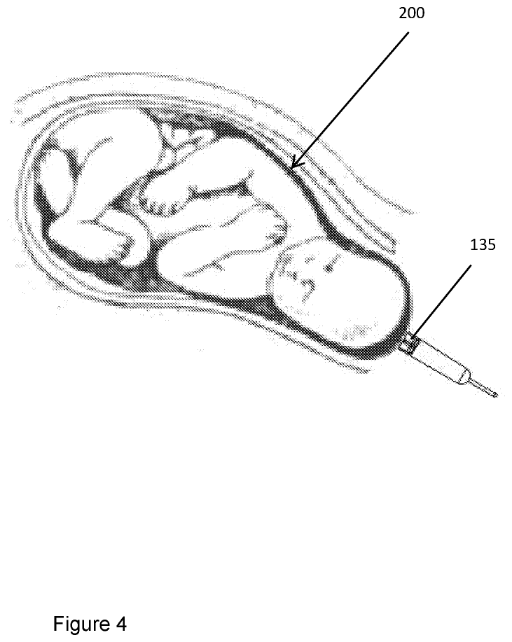

This oxy probe 100 may be used on any location on the body, and it does not have to be pressed against the tissue, for example, close (<1.0 mm) contact with the surface renders accurate data. For monitoring a fetus 200, the oxy probe 100 may be applied through a dilated cervix. The oxy probe 100 may monitor the condition of a fetus 200 during the peripartum process, measuring fetal heart rate, arterial hemoglobin oxygen saturation, electrical activity of the heart, or a combination thereof, by touching the scalp of the fetus 200 (as shown in FIG. 4) or any fetal presenting part.

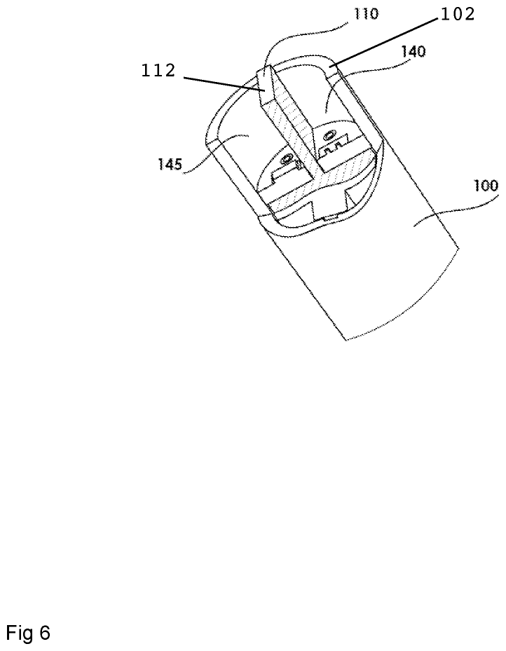

The optical divider 110 may be made of an opaque material that prevents transmission of light from reaching the sensor 125 compartment directly from the light source 130. FIG. 6 shows a cutaway view of the oxy probe tip in which it can be appreciated that the divider edge 112 extends to a height higher than the housing edge 102 (FIG. 5 also shows this, but this is harder to appreciate in FIG. 5). The housing 100 of the probe may be made of a light reflecting color such as white.

The probe tip 105, as mentioned above, may be made of optically clear soft silicon or similar materials. This clear tip 105 enables the emitted light 127 from the light source 130 to reach fetal tissue with minimal loss and allows the light to reflect back as reflected light 129 to the sensor 125 efficiently. The tip 105, being soft, also seals the probe tip such that no fluids can reach the emitter 130 and sensor 125. The sealed probe 100 can function properly to obtain accurate readings even on wet surfaces as well as when completely immersed in a fluid.

The light source 130 can include two or more light emitting diodes (LED) configured to emit light at a selected wavelength. When including more emitters, additional emitters may include a third light emitter that emits an emitted light of 550 nm to 620 nm to enable detection of other tissue bio parameters.

The detector 135 may include one or more silicon photodiodes that produce current linearly proportional to the intensity of light striking it. The detector 135 can detect the absorption and/or scattering of the light from the tissue as well as the frequency of the light emitted from the light source 130.

Unlike conventional pulse oximeters, the devices described herein need not be in direct contact with the patient's skin to obtain an accurate, consistent reading due to their being highly directional and having very high gain. As mentioned above, the oxy probe can be positioned 1 mm or less away from the skin surface and still obtain accurate oxygen saturation and heartbeat readings. The probe need not be mechanically coupled to the body to obtain an accurate reading. Because the device need not be in direct contact with the skin and there is no need for mechanical coupling to a patient, the problems that can result including pressure point injuries, pressure necrosis, exsanguinations, discomfort, compression marks, erroneous measurements, infections and other issues caused by direct contact with a device can be avoided.

### Shaped Light Sources and Detectors

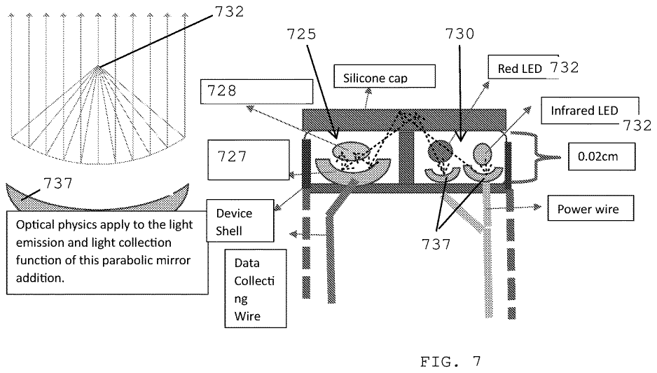

FIG. 7 shows a design with enhanced light sources 730 (red and infrared shown, though others would be possible) and detector/sensor 725. In general, this design operates similarly to the design already discussed, with light emitted from the light source 730 reflecting off tissue and the reflected light collected by the sensor 725.

The change in this design is that each light source 730 and sensor 725 includes a parabolic mirror 737, 727. The light source emitting diode 732 and sensor diode 728 are located in a focal point of each parabolic mirror 737, 727. The emitter mirrors 737 collimate light, which allows for less scatter through the tissues and therefore generates a more powerful reflection. And the reflection collection at the sensor 727 is enhanced when each collecting diode is placed at the focal point of the parabolic collector mirror 727. In use, the emitter LED 732 emits light into the emitter mirror 737 that directs light to the tissue. The sensor mirror 727 collects this reflected light from the tissue and focuses the collected light on the sensor diode 728.

The inset shows a close-up of the light source diode 732 and mirror 737 showing the light collimation. A similar diagram with reversed arrows could be shown for the collector 725.

### Oxy Probe with Camera

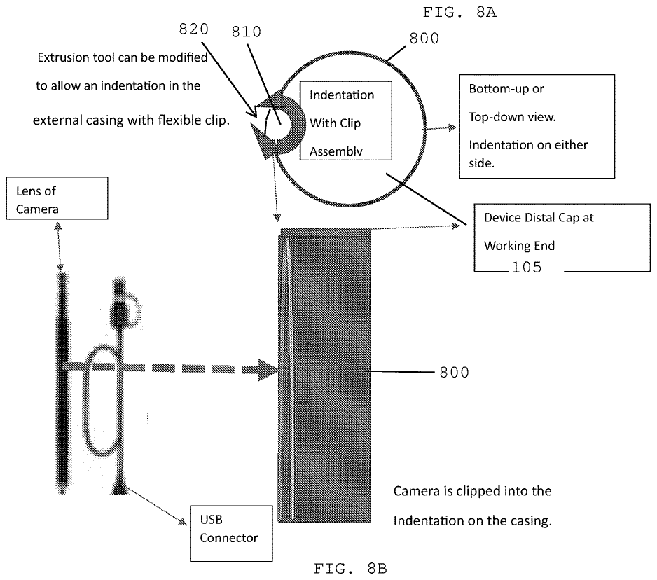

A small camera assembly 810 can be added to the external casing 800, which requires modifying the design and manufacturing of the oxy probe as previously described. In this oxy probe generally shown in FIG. 8, the external housing includes a channel or indentation 820 along its length into which a camera 810 can be inserted.

In the obstetrical use of the pulse oximeter, the oxy probe user, usually a doctor, must ensure probe placement on the skin and tissue of the presenting fetal part. For some, there may be some difficulty in palpating the uterine cervix and therefore the fetus, and this may result in false positive signals received by the pulse oximeter device. In addition, there is a relatively high incidence of mistaking maternal tissue-reflected light as fetal-reflected light. This error may result in dangerous readings.

The camera 810's placement on the pulse oximeter device allows a user, viewing a screen external to the camera and connected thereto in a wired or wireless arrangement, to view precisely what tissue the oxy probe contacts, ensuring an accurate reading during every use thereof.

### Oxy Probe with Thermocouple

The physics of temperature and oxygen transport has well established that increasing the temperature of blood results in a reduced affinity of hemoglobin for oxygen and therefore, a rightward shift of the Oxygen-Hemoglobin Dissociation curve. This is necessary in understanding the fetal body and brain temperature differences and the inherent blood-brain protective mechanisms of the fetus. The higher the blood temperature, the more hemoglobin unloads oxygen which leads to hypoxia with obvious consequences to the fetal brain.

The fetus has built-in protection for this risk and can tolerate increased temperatures in the fetal body. The fetal brain blood circulation is kept cooler, inherently, which increases its affinity for oxygen even as the rest of the blood oxygen decreases and ramps up the speed of blood-brain circulation. It has, however, been proven in the sheep in vivo model, that the longer the fetal brain-blood is subjected to heat, the quicker this protective mechanism breaks down. As a consequence the fetal brain circulation loses oxygen, which in turn leads to hypoxia and acidosis.

In an attempt to prevent this, the oxy probe housing 900 may include not only the camera 810 but also similarly in a similar indentation or channel 920, a thermocouple 910. The small, thin thermocouple 910 can be added to an indent 920 of the casing 900 of the pulse ox device, with or without the camera 810, on an ipsilateral side from the camera 810.

The necessary wires to carry power and data may be included within the channels or the housing, or each of the thermocouple and/or camera may communicate wirelessly with displays.

### Display for Oxy Probe

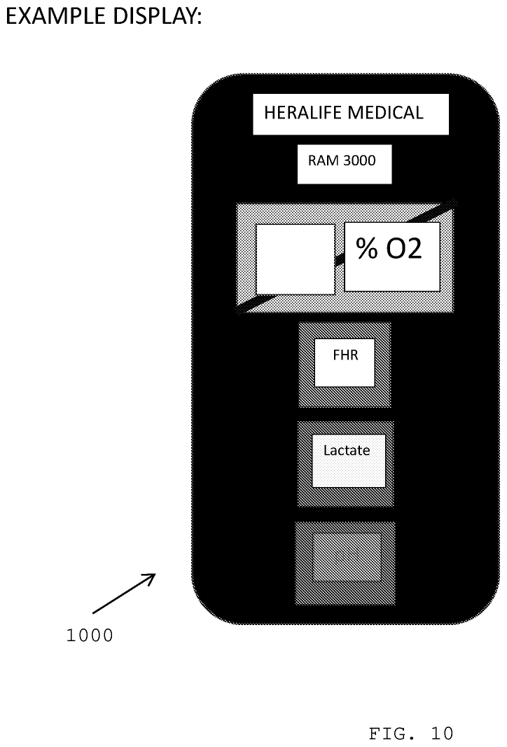

FIG. 10 shows a sample probe display 1000. The display may be non-disposable, hand-held or stationary, and it receives information from the pulse oximeter and ancillary devices in contact with the display. The data evaluation chip device may be proprietary but can include OEM productions. The display may show fSpO2, temperature, fetal heart rate (FHR), and the blood Lactate and pH as these algorithms become available.

### Wireless and Battery

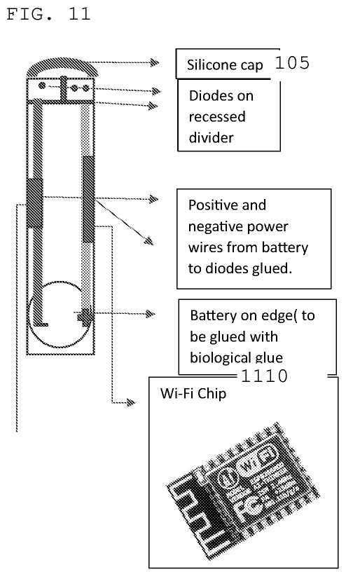

FIG. 11 shows a battery 1110 orientation in the device. The pulse oximeter described herein may be hard-wired to receive power or wireless. With a wireless setup, a battery 1110 would be required. The battery 1110, which may be a flat-round nickel-cadmium type, may be inserted inside the housing end further from the soft cap 105. The battery 1110 may be oriented in parallel with the soft cap surface that contacts the tissue, or in order to reduce the area of the housing, with its longitudinal axis at an orthogonal angle to the longitudinal axis of the housing.

As an aside, the device as shown in FIG. 11 shows the light source and sensors may be oriented on a housing wall.

### Other Applications

As already described, this device can be used in any manner of situations for the management of labor and delivery as an accurate oxygen monitor for the fetus.

The pulse ox device may also be used during craniotomy and resection of an area of the brain to detect oxygenation of tissue in apposition to the tissue removed to document that area to be unaffected by the surgery.

The device may further be used in diabetology to determine the accurate oxygenation of the affected areas of the legs, feet, and ankles.

While the invention has been described with reference to the embodiments above, a person of ordinary skill in the art would understand that various changes or modifications may be made thereto without departing from the scope of the claims.

1. An oximeter probe comprising:
a generally cylindrical housing having a distal end and a proximal end, the distal end defining a housing edge, the housing dimensioned for placement within a birth canal during a childbirth process;
wherein the housing defines a first cavity and a second cavity opening at the distal end;
the first cavity comprising at least two light emitters, the at least two light emitters comprising a first light emitter configured to emit emitted light at a first wavelength in a range from about 640 nm to about 720 nm and a second light emitter configured to emit emitted light at a second wavelength in a range from about 840 nm to about 920 nm;
the second cavity comprising a detector for detecting reflected light at both the first and second wavelengths emitted from the at least two light emitters, wherein the reflected light is the emitted light reflected off of tissue;
a divider located between the first and second cavities that prevents the emitted light from directly entering the detector, the divider having a divider edge that extends distally beyond the housing edge; and
a translucent polymer cover disposed over the distal end of the housing and extending into the first cavity and the second cavity, the translucent polymer cover configured to permit transmission of the emitted light into the tissue and transmission of the reflected light to the detector.

2. The oximeter probe of claim 1, wherein the housing is configured for intra-vaginal and intra-uterine placement.

3. The oximeter probe of claim 1, wherein the housing is dimensioned to be inserted through a dilated cervix.

4. The oximeter probe of claim 1, wherein the at least two light emitters are LED light emitters.

5. The oximeter probe of claim 1, wherein the first wavelength is in a range from about 640 nm to about 680 nm and the second wavelength is in a range from about 870 nm to about 920 nm.

6. The oximeter probe of claim 5, wherein the first wavelength is about 660 nm and the second wavelength is about 900 nm.

7. The oximeter probe of claim 1, wherein the detector comprises at least one silicon photodiode.

8. The oximeter probe of claim 1, wherein the translucent polymer cover comprises an elastomeric material.

9. The oximeter probe of claim 8, wherein the elastomeric material comprises silicone.

10. The oximeter probe of claim 1, wherein the translucent polymer cover forms a fluid-tight seal over the first cavity and the second cavity.

11. The oximeter probe of claim 1, wherein the translucent polymer cover has a substantially planar distal surface.

12. The oximeter probe of claim 1, wherein the housing is about 0.5 inches in diameter and about 2.2 inches long.

13. The oximeter probe of claim 1, wherein the oximeter probe is configured to measure oxygen saturation in the tissue from a distance of less than about 1 mm.

14. The oximeter probe of claim 1, wherein the oximeter probe is in communication with a CPU configured to determine oxygen saturation in the tissue based on a difference between the emitted light wavelength and the reflected wavelength.

15. A method for monitoring oxygen saturation of a fetus during a childbirth process, comprising:
providing an oximeter probe comprising a generally cylindrical housing having a distal end and a proximal end, the distal end defining a housing edge, the housing defining a first cavity and a second cavity opening at the distal end, the first cavity comprising at least two light emitters comprising a first light emitter configured to emit emitted light at a first wavelength in a range from about 640 nm to about 720 nm and a second light emitter configured to emit emitted light at a second wavelength in a range from about 840 nm to about 920 nm, the second cavity comprising a detector, a divider located between the first and second cavities that prevents the emitted light from directly entering the detector, the divider having a divider edge that extends distally beyond the housing edge, and a translucent polymer cover disposed over the distal end of the housing and extending into the first cavity and the second cavity;
inserting the distal end of the housing into a birth canal of a pregnant patient;
positioning the distal end such that the translucent polymer cover is in close contact with a presenting part of the fetus;
operating the oximeter probe to emit, through the translucent polymer cover, emitted light at the first wavelength and emitted light at the second wavelength, and to detect, through the translucent polymer cover, reflected light at both the first and second wavelengths reflected from the presenting part of the fetus; and
obtaining, from the oximeter probe, an oxygen saturation value of the fetus, wherein the oxygen saturation value is calculated based on a ratio of intensities of the detected reflected light at the first and second wavelengths.

16. The method of claim 15, wherein inserting comprises inserting the distal end through a dilated cervix of the pregnant patient.

17. The method of claim 15, wherein the presenting part of the fetus comprises a scalp of the fetus.

18. The method of claim 15, wherein positioning comprises positioning the distal end such that the translucent polymer cover is within about 1 mm of the presenting part of the fetus.

19. The method of claim 15, further comprising measuring a fetal heart rate of the fetus using the oximeter probe.

20. The method of claim 15, wherein the oximeter probe further comprises a camera disposed on the housing, and the method further comprises viewing, via the camera, the presenting part of the fetus to confirm that the distal end is in close contact with fetal tissue rather than maternal tissue.
